(19) United States
(12) Patent Application Publication
(10) Pub. No.: US 2010/0042113 A1
(43) Pub. Date: Feb. 18, 2010

(54) Manual odor removal structure

(76) Inventors: Pat Y. Mah, Kowloon (HK); Alexander Joseph Kalogroulis, Surrey (GB)

Correspondence Address: CURTIS L. HARRINGTON, 6300 STATE UNIVERSITY DRIVE, SUITE 250, LONG BEACH, CA, 90815, US

(21) Appl. No.: 12/228,981

(22) Filed: Aug. 18, 2008

(51) Int. Cl. A61B 17/00

(52) U.S. Cl. 606/131

(57) ABSTRACT

A volumetric stainless steel structure is provided by punch pressing a single blank and results in a volumetric presentation of alternating bands of stainless steel material. The spacing of the bands is sufficient to provide (1) an interruption in the surface to surface contact which might entrap organic species and (2) a space which allows the epidermis to be scrubbed, manipulated and massaged by the rounded edge of the bands as the hands manipulate it in a way similar the manipulation of soap. Further, the alternating bands provide internal surface area, in the form of the inside of the bands which are protruding, for additional adsorptive area.

### FIELD OF THE INVENTION

The present invention relates to an improvements in the design and operation of a manual odor remover for removing lingering unwanted food smells such as fish, onion, garlic and other objectionable smell producing molecules which tend to slightly penetrate the tissue of the hands and fingers and which are generally resistive to removal by soap and water alone, and more particularly to a stainless steel device which is shown to be more effective in odor removal.

### BACKGROUND OF THE INVENTION

If a food preparer has been working with onions, garlic, or fish long enough for the odors may be absorbed into the epidermis, the presence of stainless steel has been shown to assist in removing the odor molecules. Such smooth stainless steel rubbing objects are known to be used to help competitively adsorb odor molecules from the hands, often sold under descriptive names such as “stainless steel soap”. However, these structures have a limited surface area and do little to help manipulate the epidermis sufficient to assist in the removal of the odor producing organic molecules. Most of the recommended washing procedure involves washing the hands with soap, and while the hands are still soaped up, to manipulate a smooth stainless steel soap sized object.

The theory behind such interaction is not completely known and very likely depends upon a number of factors whose proportional contribution may vary based upon the type of molecule being removed. One suggested mechanism suggested has been an interaction between sulphur atoms in the odor molecules. The sulfur from the onion/garlic/fish might be temporarily attracted to and bind with one or more of the metals in stainless steel. Further, onions and garlic contain amino acid sulfoxides, which may form sulfenic acids, which may then form a volatile gas (propanethiol S-oxide), which may form sulfuric acid upon exposure to water. These compounds are responsible for burning eyes while food preparers cut onions. If the sulfur compounds have a preferential propensity to bind to the steel, then the odor is removed from your fingers. Where this occurs in the presence of soap, the contact of the steel with the odor molecules on the hand might be impeded, but the soap will also act to cleanse the steel as well as the hand.

A second suggested mechanism is the possibility that the stainless steel might adsorb the odor molecules, temporarily, based upon contact with the steel and the epidermis. A third mechanism, since the steel is taught to be used with water, with or without soap, is that sulfurous compounds present may either dissolve directly in the water or are catalyzed by the steel to a more soluble state. Stainless steel consists of mainly iron and chromium, and contains a thin layer of chromium (III) oxide on its surface. Metal oxides are Lewis acids and readily catalyze oxidations. Iron and chromium oxides can be used as oxidation catalysts, effective for industrial-scale oxidation of odorous reduced sulfur compounds at high temperature. A fourth plausible explanation is that a thin layer of grease, containing the odorous compounds, is rubbed off mechanically onto the steel surface. Lastly, the thin layer of Chromium(III) may also act as an adsorbent in its own right.

Most of the recommended washing procedure involves washing the hands with soap, and while the hands are still soaped up, to manipulate a smooth stainless steel soap sized object, or to manipulate the steel object and then wash the hands with soap and then repeat. In light of the aforementioned mechanisms, it is clear that contacting the hands with a smooth steel object presents a limited surface against which contact can be made. Further, absent a reaction, using a completely smooth steel object is likely to provide a significant re-sorbance onto the hands. Assuming full contact with the hands, it is easy to see why the use of “stainless steel soap” can yield results ranging from poor to average.

Further, the use of a smooth continuous surface does not assist in freeing any adsorbed molecules. An extended stainless steel surface is just as likely to enable any unreacted species (if indeed a reaction is involved) to re-sorb into the epidermis. Were the user to provide 10 or 20 cycles of rubbing contact followed by rinsing and then further rubbing in order to achieve complete effectiveness, the use of stainless steel for odor removal would be more onerous than the benefits it provides.

What is needed is a structure which will improve the effectiveness of a stainless steel object when used for removing odors from the hand. The needed structure should provide relief from re-sorbance and from trapped organic molecules resulting from extended length surface to surface contact. The needed structure should be simple to make and provide some way for the disadvantages of extended surface structures to be overcome without extended periods of washing.

### SUMMARY OF THE INVENTION

A volumetric stainless steel structure is provided from a layer which results in an volumetric presentation of alternating bands of stainless steel material. The spacing of the bands is sufficient to provide (1) an interruption in the surface to surface contact which might entrap organic species and (2) a space which allows the epidermis to be scrubbed, manipulated and massaged by the rounded edge of the bands as the hands manipulate it in a way similar the manipulation of soap. Further, the alternating bands provide internal surface area, in the form of the inside of the bands which are protruding, for additional adsorptive area.

The massaging/scrubbing action of the structure, combined with an area roughly equivalent to a soap bar, but with half the area being freed from trapped enclosing entrainment, will enable at least an equivalent adsorbance with respect to a conventional “stainless steel soap”. However, the action of the interstitial space between the bands, in allowing for (1) relief from enclosed entrapment of the organic species, (2) massaging of the skin to help any epidermis absorbed/adsorbed species to be moved, (3) a differentially stronger and yet less noticible scraping, and (4) an internal flow-through design, is expected to optimize the effectiveness in removing organic material from the hands.

The internal space formed by the alternating bands is easily accessible by brush in order to “clean” the removal structure. Further, although it will not require cleaning either externally or internally due to its soap and water environment, and the fact that it is constantly used in cleaning, it can be easily cleaned in a number of ways. Any hard water sediments can be removed by a reducing agent. The inside of the odor removal structure, as well as the outside can be accessed for cleaning by a brush, autoclave, dishwasher, or by soaking in harsher chemicals such as clorox or other sterilization agents.

Further, the appearance of the odor removal structure can be highly modified by altering the surface finish. A roughened finish can provide more abrasiveness in the contact of the user's epidermis with the surface of the outside alternating bands, and provide the most help in dislodging any odor carrying organic molecules held in the outermost epidermis. A roughly brushed finish will provide increased surface area, perhaps less than a roughened finish. A highly polished finish will provide less help in dislodging molecules from the epidermis, but will provide a more attractive finish.

Further, because the odor removal structure has a significant visual profile and significant external, and internal surface, it is amenable to even more complicated and pleasing designs being imposed, as well as text writing into the material, without any loss of active surface area, such as advertising, trademarks, indications of source, and other information, including instructions for use. Further, the odor removal structure has an outer rim common to both sets of lateral structures and which can also carry writing, instructions and further designs. Further, the design illustrated can be widely varied in terms of its appearance and still produce equivalent effects. For example, a female user with smaller hands and softer epidermis might prefer a model having narrower bands with less open space than a man with larger hands and a more rigid epidermis. Soft skin would have a greater tendency to enter the interstitial spaces and be massaged and manipulated by the adjacent bands.

### BRIEF DESCRIPTION OF THE DRAWINGS

The invention, its configuration, construction, and operation will be best further described in the following detailed description, taken in conjunction with the accompanying drawings in which:

FIG. 1 is a perspective view illustrating the exterior overall shape of the odor removal structure and enables an explanation of the exterior structures; and

FIG. 2 is a view taken along line 2-2 of FIG. 1 and illustrating the internal surfaces and structures of the odor removal structure seen in FIG. 1.

### DETAILED DESCRIPTION OF THE PREFERRED EMBODIMENT

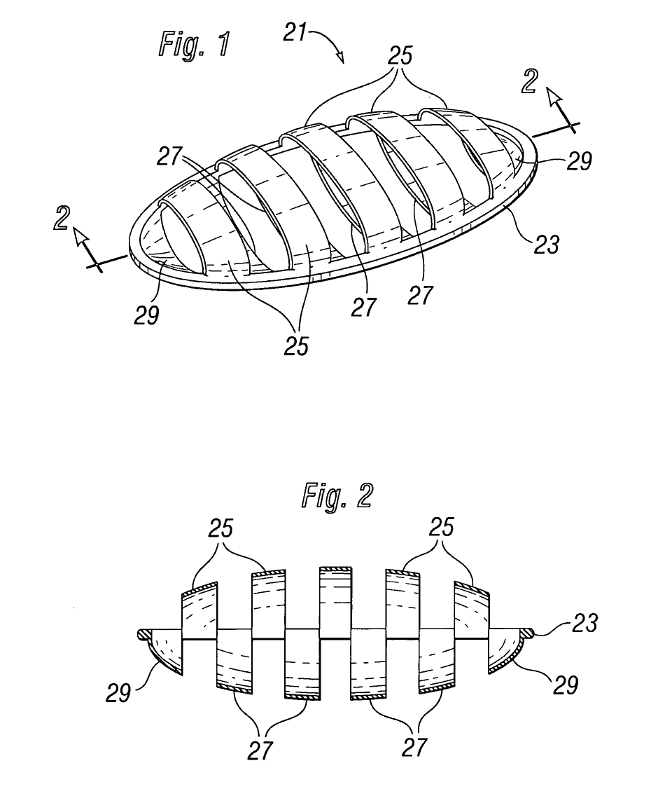

The description and operation of the odor removal structure 21 of the invention will be best described with reference to FIG. 1 which illustrates a single structure which may preferably be stamped from a single sheet of formed material. The odor removal structure 21 has an outer rim 23 which may be generally oval shaped. Outer rim 23 supports a series of alternating lateral bands which include first side bands 25, each of which has two ends, and where each of the two ends are attached to the outer rim 23 and punched from the outer rim 23. A series of second side bands 27 which are continuous with the outer rim 23, but punched in the opposite direction are also partially seen through the spaces separating the first side bands 25. The terminal punched structures 29 happen to be extending to the same side as the second side bands and have one free edge, with the opposite edge being continuous with the ends of the outer rim 23 near the oval ends of the overall odor removal structure 21.

Counting the total number of side bands 25 and 27 and terminal punched structures 29, they total eleven as seen in FIG. 1. Ideally, the odor removal structure 21 may be from about five to ten centimeters in overall length and with a width about half of the overall length and a height of from about one and a half to about three and a half centimeters. The odor removal structure 21 may be preferably formed from a stainless steel blank having an oval shape and a metal thickness of about 0.5 millimeters. The side bands 25 and 27 and terminal punched structures 29 can be formed with a single punching operation.

Referring to FIG. 2, a view taken along line 2-2 of FIG. 1 is a sectional view which gives a good view of the internal space and illustrates how each of the side bands 25 and 27 are punched, and produces a general ninety degree angle between the outer surface and lateral punched surface, limited only slightly by the rising and declining profile across the length of the odor removal structure 21. The absolute edge of this angle may be micro-softened by tumbling, but the edge is available to help scrape the hands in a safe way. The eleven segments thus have a width of a little over a half centimeter, and this also translates into interstitial spaces or openings between the side bands 25 and 27 and terminal punched structures 29 of the same dimension. The bands can be of uneven width, can be of a wider width to result in more interstitial space or can be of narrower width. The odor removal structure 21 is typically tumbled and de-burred so that the edges of the bands 25 & 27 will not be sharp enough to cut the user, but will be angled sufficiently to scrub and massage. As the user rubs the odor removal structure 21, the user's epidermis rubs over the bands 25 & 27 and terminal punched structures 29. As such there occurs direct contact, rubbing, massaging, scraping and a general exercising of the epidermis which should be more than sufficient to free the hands of the organic odor molecules by any number of the mechanism described above. The user has the option, rather than having to repeatedly wash, to simply wash harder by pressing harder on the odor removal structure 21. Unlike a smooth stainless steel structure, the user might have to resort to other non-stainless steel structures, such as sponges and scrubbing pads and the like, in order to exercise and manipulate the epidermis. Such other structure manipulation would not have the benefits of contact with the stainless steel while occurring. Depending upon which mechanism works best with the odor being removed, not having the ability to scrub and massage the epidermis with the stainless steel will not produce good results.

While the present invention has been described in terms of a structure, particularly a stainless steel structure for use in cleaning the hands and epidermis of odor containing molecules, and using a variety of mechanisms, any one or group of which may predominate in reacting and assisting removal depending on the type of molecule being removed, one skilled in the art will realize that the structure and techniques of the present invention can be applied to many structures and devices which are used for cleaning, massaging, and reactively removing molecules.

Although the invention has been derived with reference to particular illustrative embodiments thereof, many changes and modifications of the invention may become apparent to those skilled in the art without departing from the spirit and scope of the invention. Therefore, included within the patent warranted hereon are all such changes and modifications as may reasonably and properly be included within the scope of this contribution to the art.

What is claimed:

1. An odor removal structure comprising:
a stainless steel body having a first side having a series of bands separated by a series of interstitial spaces for enabling the epidermis to be rubbed over the bands to make contact with odor molecules and to massage and scrape past the epidermis to assist in removing odor bearing molecules, said body having a second side.

2. The odor removal structure as recited in claim 1 wherein the body has an outer rim from which the bands depend.

3. The odor removal structure as recited in claim 2 and wherein said body has a terminal punched structure extending from the rim to the beginning of said interstitial space.

4. The odor removal structure as recited in claim 1 wherein the bands extend parallel to each other.

5. The odor removal structure as recited in claim 1 wherein the second side of said body also has a series of bands and for two sided scrubbing.

6. The odor removal structure as recited in claim 1 wherein said body is formed from a single expanse of material.

7. The odor removal structure as recited in claim 1 wherein said body is formed from stamping a metal blank.

8. The odor removal structure as recited in claim 5 wherein both said first side and said second side bands are formed from stamping a metal blank.
